(19) United States
(12) Patent Application Publication
(10) Pub. No.: US 2025/0216367 A1
(43) Pub. Date: Jul. 3, 2025

(54) HYDROGEN SULFIDE TEST KIT AND METHOD FOR DETECTING HYDROGEN SULFIDE IN AN AQUEOUS SAMPLE

(71) Applicant: HACH COMPANY, Loveland, CO (US)

(72) Inventors: Melinda BUYCK, Nevada, IA (US); Emily POWELL, Loveland, CO (US)

(73) Assignee: HACH COMPANY, Loveland, CO (US)

(21) Appl. No.: 18/398,735

(22) Filed: Dec. 28, 2023

(51) Int. Cl. G01N 31/22; G01N 33/18

(52) U.S. Cl. CPC G01N 31/224; G01N 33/18

(57) ABSTRACT

A hydrogen sulfide test kit includes a first reactant powder that includes citric acid, a second reactant powder that includes sodium bicarbonate, and a test paper that changes color upon contact with hydrogen sulfide. The first and second reactant powders are physically separated from each other. A method of using the hydrogen sulfide test kit and a method for detecting hydrogen sulfide in an aqueous sample bot include allowing the citric acid and the sodium bicarbonate to react in the aqueous sample to form air bubbles that release any hydrogen sulfide in the aqueous sample into gas form; contacting the released hydrogen sulfide in gas form, if present, with the test paper; and detecting the presence of hydrogen sulfide in the aqueous sample based on the color of the test paper.

### BACKGROUND

Hydrogen sulfide (H₂S) is a common contaminant of water sources that requires monitoring. Hydrogen sulfide is formed when soluble sulfides are hydrolyzed in water. In water, hydrogen sulfide dissociates, forming monohydrogensulfide (1-) (HS⁻) and sulfide (S²⁻) ions.

Hydrogen sulfide may be produced by bacteria, or released from naturally occurring geologic deposits. For example, the presence of hydrogen sulfide in a water sample can be indicative of microbial activity or volcanic gas production. Thus, its presence can reflect the existence of other underlying problems.

Additionally, hydrogen sulfide is highly corrosive and can lead to premature damage and failure of equipment. Corrosion caused by hydrogen sulfide increases production well operating costs significantly when equipment is damaged and needs replacing.

Hydrogen sulfide is also toxic, even in very low amounts. Once ingested, it will form a complex with the iron (III) ion of the mitochondrial metalloenzyme cytochrome oxidase, blocking oxidative metabolism. It also alters or inhibits many other enzymes, rapidly leading to acute complications.

Exposure to minimal amounts of hydrogen sulfide can range from more minor symptoms of irritation (e.g., irritation of the eyes and respiratory tract, nausea, vomiting, epigastric pain) at amounts ranging from 20 to 250 ppm, to more severe symptoms impacting the nervous system (unconsciousness and respiratory paralysis) at amounts higher than 250 ppm. The National Institute for Occupational Safety and Health (NIOSH) considers hydrogen sulfide concentrations of 100 ppm to be immediately dangerous to life or health. Concentrations greater than 500 ppm can cause a person to collapse within five minutes, and concentrations exceeding 700 ppm can cause immediate collapse, and death, within just one or two breaths. Accordingly, the Occupational Safety and Health Administration (OSHA) regulates that the permissible exposure limit for hydrogen sulfide is 20 ppm.

Although hydrogen sulfide has a recognizable odor likened to rotten eggs, this odor is only perceptible at very low concentrations (less than 10 ppm in air). Beyond this, the odor can be deceptively sweet, and higher concentrations will deaden the sense of smell. This means that someone can be exposed to potentially toxic amounts of hydrogen sulfide without there being a detectable smell.

Current methods for detecting hydrogen sulfide in aqueous samples are generally limited to offsite testing applications. For example, hydrogen sulfide is traditionally detected using an acid displacement procedure in which the hydrogen sulfide is displaced by acidification and detected by gas chromatography. So-called “dip strip” techniques, although more portable, have low accuracy.

Thus, there is a need to quickly and easily test for hydrogen sulfide in aqueous samples from sources such as wastewater, drinking water, and well water. In order to do so, the test should be accurate at detecting low concentrations of hydrogen sulfide, and be capable of being performed in situ (at the site of the water source).

### SUMMARY

The disclosed embodiments describe a hydrogen sulfide test kid, a method of using the hydrogen sulfide test kit, and a method for detecting hydrogen sulfide in an aqueous sample.

According to one aspect of the embodiments, a hydrogen sulfide test kit includes a first reactant powder comprising citric acid; a second reactant powder comprising sodium bicarbonate; and a test paper configured to change color upon contact with hydrogen sulfide. The first and second reactant powders are physically separated from each other.

Another aspect of the embodiments includes a method of using the hydrogen sulfide test kit. The method includes adding the first and second reactant powders to an aqueous sample; allowing the citric acid and the sodium bicarbonate to react in the aqueous sample to form air bubbles, wherein the air bubbles release any hydrogen sulfide in the aqueous sample into gas form; contacting the released hydrogen sulfide in gas form, if present, with the test paper; and detecting the presence of hydrogen sulfide in the aqueous sample based on the color of the test paper.

Another aspect of the embodiments includes a method for detecting hydrogen sulfide in an aqueous sample. The method includes adding citric acid and sodium bicarbonate to the aqueous sample; allowing the citric acid and the sodium bicarbonate to react in the aqueous sample to form air bubbles, wherein the air bubbles release any hydrogen sulfide in the water sample into gas form; contacting the released hydrogen sulfide in gas form, if present, with a test paper that is configured to change color upon contact with the hydrogen sulfide; and detecting the presence of hydrogen sulfide in the aqueous sample based on the color of the test paper. In this method, the citric acid and the sodium bicarbonate have not been in contact with each other for more than 24 hours before adding the citric acid and the sodium bicarbonate to the aqueous sample.

### BRIEF DESCRIPTION OF THE DRAWINGS

A more particular description of the invention will be rendered by reference to specific embodiments thereof that are illustrated in the appended drawings. Understanding that these drawings depict only typical embodiments of the invention and are not therefore to be considered to be limiting of its scope, embodiments of the invention will be described and explained with additional specificity and detail through the use of the accompanying drawings in which:

FIG. 1 is a flow chart illustrating a method of detecting hydrogen sulfide in an aqueous sample according to the disclosed embodiments; and

FIG. 2 is a schematic representation of a hydrogen sulfide test chart of the disclosed embodiments.

### DETAILED DESCRIPTION OF EMBODIMENTS

Existing methods for testing for hydrogen sulfide in aqueous samples generally require offsite analysis or can only determine the amount of hydrogen sulfide with low accuracy. Thus, there is a need for a kit that would allow for onsite testing for hydrogen sulfide in an aqueous sample with high accuracy and resolution.

The inventors found that hydrogen sulfide could be detected and quantified with high accuracy in an aqueous sample by releasing the hydrogen sulfide into gas form and detecting the hydrogen sulfide in its gas form.

For example, effervescent tablets such as Alka-Seltzer Gold™ could be used to form air bubbles in the aqueous sample. The tablets include sodium bicarbonate (NaHCO₃) and citric acid (HOC (CH₂CO₂H)₂)—a base and an acid, respectively. When the tablet is dissolved in water, bicarbonate (HCO₃⁻) and hydrogen ions (H⁺) are formed. Once in solution, the bicarbonate and hydrogen ions react according to the reaction shown below.

HCO₃⁻ (aq)+H⁺(aq)→H₂O (l)+CO₂ (g)

The carbon dioxide bubbles to the surface, allowing any hydrogen sulfide in the aqueous sample to be released in gas form. Specifically, it is believed that the hydrogen sulfide is released by the shift in pH caused by the reaction, and the effervescence brings the hydrogen sulfide out of solution.

Once in gas form, the hydrogen sulfide could then be permitted to contact a test paper that is configured to change color upon contact with hydrogen sulfide. It was found that even very small amounts of hydrogen sulfide could be detected with high resolution (e.g., 0.1 or 0.2 ppm differences in concentration) when detected in gas form. By contrast, similar methods for detecting hydrogen sulfide in liquid form using color-changing paper (so-called “dip strip” methods) do not allow for distinction between concentrations with differences of less than about 2 ppm.

Despite this improvement, the method's reliance on effervescent tablets was found to be disadvantageous. By combining the reactive ingredients in tablet form, the method did not allow for any adjustment in the amount of reactive ingredients. Thus, the method could not be easily adapted for different applications.

Additionally, the tablets contained other ingredients in order to prevent premature reaction of the active ingredients. For example, Alka-Seltzer Gold™ tablets contain citric acid and sodium bicarbonate as active ingredients, potassium bicarbonate as a stabilizer, and magnesium stearate as a desiccant. Without the stabilizer and desiccant, the citric acid and sodium bicarbonate would begin to react with each other. Thus, the reactive ingredients cannot be packaged together without requiring additional filler ingredients. These filler ingredients add to cost and bulk and have the potential to interfere with the reaction process and reduce testing accuracy.

The inventors conducted numerous studies and found that hydrogen sulfide could be detected and quantified with high accuracy in an aqueous sample by releasing the hydrogen sulfide into gas form using citric acid and sodium bicarbonate in powder form, and then detecting the hydrogen sulfide in its gas form. The citric acid and sodium bicarbonate can be stored separately in powder form to prevent them from reacting with each other, and to allow for precise adjustment of reaction amounts depending on the circumstances of the test sample.

In particular, the disclosed embodiments include a hydrogen sulfide test kit that includes citric acid and sodium bicarbonate. For example, the citric acid and sodium bicarbonate can be isolated from each other in separate containers until ready for use.

In one embodiment, a first container includes a first reactant powder comprising citric acid, and a second container includes a second reactant powder comprising sodium bicarbonate. In order keep the citric acid and sodium bicarbonate isolated from each other so that they do not react with each other, the first reactant powder is substantially free of sodium bicarbonate, and the second reactant powder substantially free of citric acid. Herein, “substantially free of” means that the component is contained in amounts that do not significantly affect the reaction chemistry. For example, because the first reactant powder is substantially free of sodium bicarbonate, any sodium bicarbonate present in the first reactant powder would react with the citric acid to a negligible extent. In one embodiment, the first reactant powder does not include any sodium bicarbonate, and the second reactant powder does not include any citric acid.

The first and second reactant powders can optionally include other non-reactive ingredients. For example, the first and second reactant powders can include a stabilizer and/or a desiccant. Alternatively, the first and second reactant powders can be free of any stabilizer or desiccant. In one embodiment, the first reactant powder consists of citric acid, and the second reactant powder consists of sodium bicarbonate.

The citric acid and the sodium bicarbonate are substantially pure (meaning that they contain less than 5 wt % of impurities). For example, the citric acid and the sodium bicarbonate contain less than 3 wt %, preferably less than 1 wt %, and more preferably less than 0.5 wt % of impurities.

The sodium bicarbonate can have a particle size in the range of 50 to 600 μm, 75 to 500 μm, 100 to 400 μm, or 200 to 300 μm, for example.

The kit also includes a test paper configured to change color upon contact with hydrogen sulfide. For example, the test paper can include copper sulfate, which will react with hydrogen sulfide to form copper sulfide according to the following reaction.

CuSO₄+H₂S→CuS+H₂SO₄

The copper sulfate may be coated on a surface of the test paper in order to ensure contact with the hydrogen sulfide.

The quantity of reaction product will depend on the amount of hydrogen sulfide that comes into contact with the test paper, which in turn depends on the amount of hydrogen sulfide present in the aqueous sample. A larger amount of reaction product can be visualized as a change in color. Thus, the test paper allows for both the detection and quantification of hydrogen sulfide in the aqueous sample.

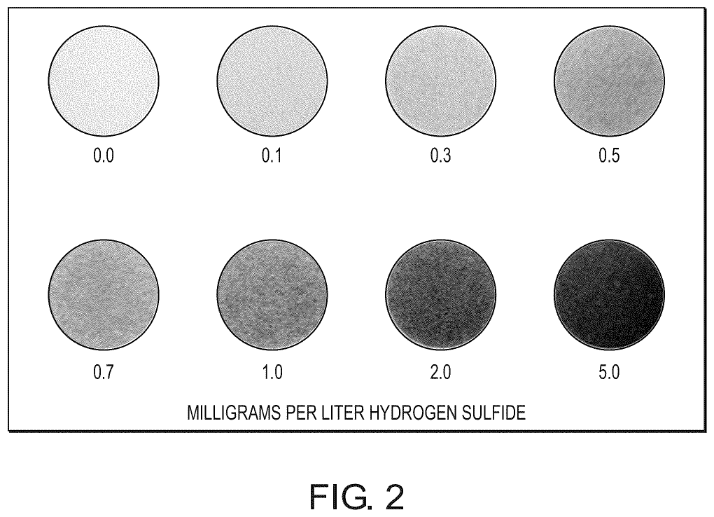

In order to allow for quantification of hydrogen sulfide, the test kit also includes a test chart. An example of a test chart of the disclosed embodiments is shown in FIG. 2. As shown in FIG. 2, the color of the test paper gets progressively darker as the detected amount of hydrogen sulfide increases. By visually comparing the test paper to the test chart, it is possible to determine the approximate amount of hydrogen sulfide in the aqueous sample. For example, if the test paper is approximately the same color as the example for 0.3 ppm shown on the test chart, then the amount of hydrogen sulfide in the aqueous sample is determined to be approximately 0.3 ppm. If the coloration is midway between that shown for the 0.3 ppm and 0.5 ppm on the test chart, then the amount of hydrogen sulfide in the aqueous sample is determined to be between 0.3 ppm and 0.5 ppm (for example, approximately 0.4 ppm). In this way, differences as little as about 0.03 ppm can be distinguished. Approximations can be accurate up to +0.03 ppm.

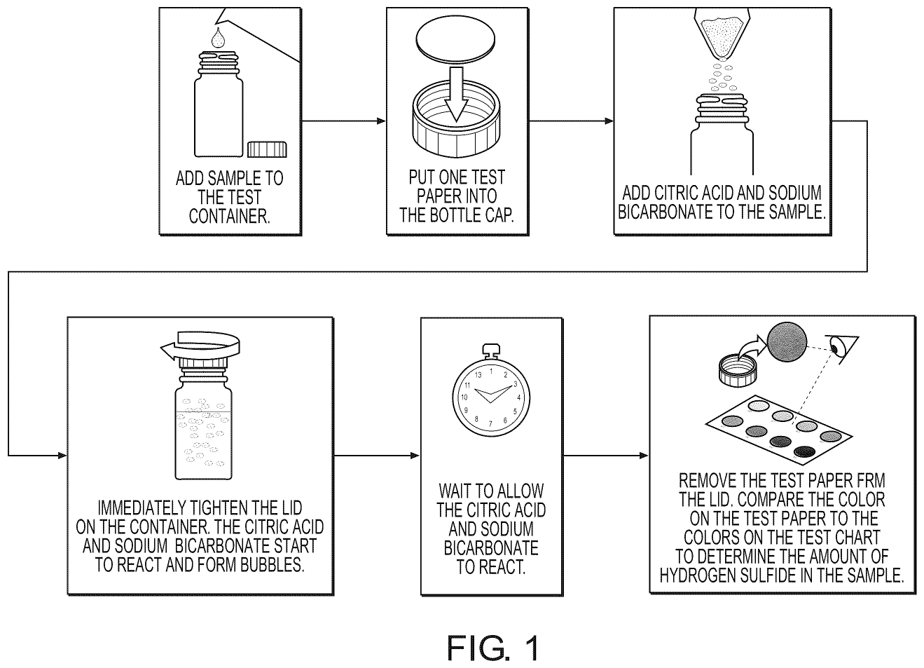

The disclosed embodiments also relate to a method for detecting hydrogen sulfide in an aqueous sample, as summarized in FIG. 1. When performing the hydrogen sulfide test method, the citric acid and sodium bicarbonate should remain physically separated from each other until ready for use in order to avoid premature reaction, which could reduce the efficacy of the test. In some instances, it might be possible to combine the citric acid and sodium bicarbonate in advance and store the mixture in a low-moisture environment (for example, at less than 10% relative humidity at 23° C., or under vacuum). However, such exposure should be limited to less than 24 hours in advance of conducting the test, and preferably should be avoided altogether.

Preferably the citric acid and sodium bicarbonate are not combined with each other until immediately before conducting the test. For example, the citric acid and the sodium bicarbonate are in contact with each other for 15 minutes or less before adding the citric acid and the sodium bicarbonate to the aqueous sample for testing. Preferably the citric acid and the sodium bicarbonate are in contact with each other for 10 minutes or less, 5 minutes or less, or 1 minute or less before adding the citric acid and the sodium bicarbonate to the aqueous sample. In an embodiment, the citric acid and sodium bicarbonate are separately added to the aqueous sample, and thus are not combined in advance of being added to the aqueous sample.

The aqueous sample for testing may be a water sample such as wastewater, drinking water, or well water, for example. The aqueous sample comprises at least 95 wt %, 98 wt %, 99 wt %, or 99.8 wt % water.

The aqueous sample may contain hydrogen sulfide. The hydrogen sulfide content in the aqueous sample may be 10.0 mg H₂S/L sample (i.e., 10.0 ppm H₂S) or less, 5.0 ppm or less, 2.0 ppm or less, 1.0 ppm or less, 0.7 ppm or less, 0.5 ppm or less, 0.3 ppm or less, or 0.1 ppm or less. The hydrogen sulfide content in the aqueous sample may be 5.0 ppm or more, such as 8 ppm, 10 ppm, 15 ppm, or 20 ppm, for example.

In order to improve the accuracy of the testing, the aqueous sample should ideally be maintained at a temperature below 50° C. during testing. For example, the temperature can be in the range of from 5° C. to 40° C., 10° C. to 37° C., 12° C. to 30° C., or 10° C. to 23° C.

The aqueous sample can have a pH in the range of from 3.7 to 9.7. For example, the aqueous sample may have a pH in the range of from 4.0 to 9.5, 5.0 to 8.0, or 6.5 to 7.5.

The aqueous sample can have a hardness of 0 to 180 ppm calcium carbonate (CaCO₃). For example, the aqueous sample may have a hardness in the range of from 10 to 150 ppm, 25 to 130 ppm, or 50 to 100 ppm.

As shown in FIG. 1, the test method includes adding an aqueous sample to a test container. The volume of aqueous sample added to the test container can range from 50 500 mL, 75 to 200 mL, or 100 to 150 mL, for example. The test container should be impermeable and chemically inert so as not to interfere with the testing process. For example, the test container can be formed of glass. Once the aqueous sample is collected and added to the container, it should be analyzed immediately in order to ensure that the measurements accurately reflect hydrogen sulfide concentrations in the source of the aqueous sample.

Next, a test paper is positioned in proximity to the aqueous sample so that any hydrogen sulfide released from the aqueous sample will contact with the test paper. For example, the test paper is positioned inside the test container, such as inside the lid. The test paper can be positioned inside the lid so that it is in fitted arrangement with the lid and remains fixed inside the lid when the lid closes the opening of the test container, as shown in FIG. 2. The test paper can be removed from the lid after the test is completed so that the container can be washed and reused with a new test paper.

Once the test paper is positioned inside the container (for example, inside the lid), the first and second reactant powders are measured and added to the aqueous sample inside the container. The first and second reactant powders can be added together or separately, as discussed above. A total amount of the sodium bicarbonate and citric acid added to the aqueous sample can be in the range of from 1 to 8 mg per 100 mL of the sample. For example, the total added amount can be in the range of 2 to 6 mg, or 3 to 4 mg.

Depending on the source of the aqueous sample, the amounts of the first and second reactant powders that are added to the aqueous sample can be adjusted as desired. For example, the first and second reactant powders can be added so that a ratio of sodium bicarbonate to citric acid added to the aqueous sample is 30-70:70-30. For example, the ratio can be 35:65, 40:60, 45:55, 50:50, 55:45, 60:40, or 65:35.

As mentioned above, the aqueous sample should be analyzed as soon as practically possible after it has been collected in order to ensure that the measurements accurately reflect hydrogen sulfide concentrations in the source of the aqueous sample. Accordingly, the first and second reactant powders should be added to the aqueous sample within 2 hr of collecting the aqueous sample, and preferably within 1 hr, 30 min, 15 min, 10 min, or 5 min or less.

After adding the first and second reactant powders, the container is closed (for example, by closing the lid) and the first and second reactant powders are allowed to react, creating bubbles. The reaction is allowed to progress for 30 s to 10 min, 45 s to 5 min, 1 min to 4 min, or 1.5 min to 2 min, for example. During this time, the container can be agitated as needed to ensure that the reaction runs to completion. For example, the container can be gently moved so that the contents swirl inside the container, mixing the first and second reactant powders with the aqueous sample. It is expected that all of the hydrogen sulfide in a 100 mL sample will be released from solution within approximately 2 minutes.

After waiting for a period of time, the test paper is removed from the container and visually observed. The observations can be done with the naked eye or with magnification. If visual observation reveals that the test paper has undergone a color change, then it is determined that hydrogen sulfide is present in the aqueous sample. The test paper can be compared to the test chart to confirm the presence of hydrogen sulfide and optionally quantify the amount of hydrogen sulfide present in the aqueous sample. Visual observation is preferably performed within 15 minutes of removing the test paper from the container. For example, the test paper is visually observed 10 minutes or less, 5 minutes or less, 2 minutes or less, or 1 minute or less after removing it from the reaction conditions.

Modifications if this test method can be made. For example, the test paper can be added to the container before adding the aqueous sample, after adding the aqueous sample but before adding the first and second reactant powders, or even after adding the first and second reactant powders if done quickly. The test can be performed over a range of environmental conditions, such as a relative humidity in the range of from 20 to 90%, 30 to 80%, or 40 to 60%.

It will be appreciated that the above-disclosed features and functions, or alternatives thereof, may be desirably combined into different kits and methods. Also, various alternatives, modifications, variations or improvements may be subsequently made by those skilled in the art, and are also intended to be encompassed by the disclosed embodiments. As such, various changes may be made without departing from the spirit and scope of this disclosure.

What is claimed is:

1. A hydrogen sulfide test kit comprising:
a first reactant powder comprising citric acid;
a second reactant powder comprising sodium bicarbonate; and
a test paper configured to change color upon contact with hydrogen sulfide,
wherein the first and second reactant powders are physically separated from each other.

2. The hydrogen sulfide test kit of claim 1, wherein:
the first reactant powder is substantially free of sodium bicarbonate; and
the second reactant powder is substantially free of citric acid.

3. The hydrogen sulfide test kit of claim 1, further comprising:
a first reactant container containing the first reactant powder; and
a second reactant container containing the second reactant powder.

4. The hydrogen sulfide test kit of claim 3, wherein:
the first reactant container contains only the first reactant powder; and
the second reactant container contains only the second reactant powder.

5. The hydrogen sulfide test kit of claim 1, wherein the first and second reactant powders do not include any desiccant.

6. The hydrogen sulfide test kit of claim 1, wherein the first and second reactant powders do not include any stabilizer.

7. The hydrogen sulfide test kit of claim 1, wherein the first and second reactant powders do not include any potassium bicarbonate.

8. The hydrogen sulfide test kit of claim 1, wherein the test paper comprises copper sulfate.

9. The hydrogen sulfide test kit of claim 1, wherein a degree to which the test paper changes color upon contact with hydrogen sulfide depends on an amount of the hydrogen sulfide that contacts with the test paper.

10. The hydrogen sulfide test kit of claim 1, further comprising:
a test container configured to hold an aqueous sample, the test container comprising:
an opening; and
a removable lid configured to:
close the opening of the test container, and
hold the test paper such that the test paper is exposed to an inside of the test container when the lid closes the opening of the test container.

11. The hydrogen sulfide test kit of claim 1, further comprising:
a test chart including color-coded results for a variety of concentrations of hydrogen sulfide.

12. A method of using the hydrogen sulfide test kit of claim 1, the method comprising:
adding the first and second reactant powders to an aqueous sample;
allowing the citric acid and the sodium bicarbonate to react in the aqueous sample to form air bubbles, wherein the air bubbles release any hydrogen sulfide in the aqueous sample into gas form;
contacting the released hydrogen sulfide in gas form, if present, with the test paper; and
detecting the presence of hydrogen sulfide in the aqueous sample based on the color of the test paper.

13. The method of claim 12, wherein:
the hydrogen sulfide test kit further comprises a test chart including color-coded results for a variety of concentrations of hydrogen sulfide, and
the presence of hydrogen sulfide in the aqueous sample is detected by visually comparing the color of the test paper to the color-coded results.

14. A method for detecting hydrogen sulfide in an aqueous sample, the method comprising:
adding citric acid and sodium bicarbonate to the aqueous sample;
allowing the citric acid and the sodium bicarbonate to react in the aqueous sample to form air bubbles, wherein the air bubbles release any hydrogen sulfide in the aqueous sample into gas form;
contacting the released hydrogen sulfide in gas form, if present, with a test paper that is configured to change color upon contact with the hydrogen sulfide; and
detecting the presence of hydrogen sulfide in the aqueous sample based on the color of the test paper,
wherein the citric acid and the sodium bicarbonate have not been in contact with each other for more than 24 hours before adding the citric acid and the sodium bicarbonate to the aqueous sample.

15. The method of claim 14, wherein the citric acid and the sodium bicarbonate have not been in contact with each other for more than 15 minutes before adding the citric acid and the sodium bicarbonate to the aqueous sample.

16. The method of claim 14, wherein the citric acid and the sodium bicarbonate are added to the aqueous sample separately from each other.

17. The method of claim 14, wherein the presence of hydrogen sulfide in the aqueous sample is detected by visually observing the color of the test paper.

18. The method of claim 14, wherein:
the citric acid is added to the aqueous sample in the form of a first reactant powder that is substantially free of sodium bicarbonate; and
the sodium bicarbonate is added to the aqueous sample in the form of second reactant powder that is substantially free of citric acid.

19. The method of claim 18, wherein:
the first reactant powder is added to the aqueous sample from a first reactant container; and
the second reactant powder is added to the aqueous sample from a second reactant container.

20. The method of claim 19, wherein:
the first reactant container contains only the first reactant powder; and
the second reactant container contains only the second reactant powder.
